(19) United States
(12) Patent Application Publication
(10) Pub. No.: US 2025/0288332 A1
(43) Pub. Date: Sep. 18, 2025

(54) Scoliosis Correction Device as Suspended on Spinous Processes

(71) Applicants: Cheng Ching Yeh Wang, Taipei City (TW); Paonan Biotech Co., Ltd., Taipei City (TW)

(72) Inventor: Chung Chun Yeh, Taipei City (TW)

(21) Appl. No.: 18/960,100

(22) Filed: Nov. 26, 2024

(30) Foreign Application Priority Data
Mar. 12, 2024 (TW) 113108891

(51) Int. Cl. A61B 17/70

(52) U.S. Cl. CPC A61B 17/7065; A61B 17/7068

(57) ABSTRACT

A scoliosis correction device comprises: a first bracket relying on a first side of a spinous process, and having a first socket formed on the first bracket; a second bracket relying on a second side of the spinous process and having a second socket formed on the second bracket, and having at least a fastening member provided for fastening the first bracket and the second bracket on a spinous process; a first guiding member connected with the first socket having at least a first guiding hole formed through the first guiding member for slidably inserting a first correction member through the first guiding hole; and a second guiding member connected with the second socket, having at least a second guiding hole formed through the second guiding member for slidably inserting a second correction member through the second guiding hole.

### BACKGROUND OF THE INVENTION

In a traditional growing rod (TGR), the growing rod consists of proximal and distal anchors of pedicle screws attached to the spine, and two vertical rods attached to the archors. The distal and proximal rods are connected by a telescopic unit for distraction or lengthening for correcting the deformed spine. The lengthening procedure is performed every six months, involving a surgical incision in the patient's back. Such repeated surgical lengthenings significantly increase the risk of complications. Also, it may cause pain either physically or psychologically to the patient.

MCGR (Magnetically-Controlled Growing Rods) had been achieved without the need for invasive surgical lengthening. Magnetically-controlled growing rods are adjustable rods placed next to the spine during surgery. The rods expand by using magnets and an external remote controller. Even the MCGR may overcome the drawbacks of repeated surgical lengthenings as required by TGR, the patient, due to his or her continuing growth, the spine still requires frequent adjustments, along with the patient's growth. Such adjustments of MCGR must be manipulated by a professional doctor, being unable to be automatically adjusted, and thereby still causing inconvenience for the patient.

U.S. patent application Ser. No. 18/656,738, also filed by the same Applicant of this application, disclosed a scoliosis correction device comprising: a plurality of saddles each having a guiding hole formed through the saddle, and secured to a pedicle; and at least a correction rod slidably engageable within said guiding hole in each said saddle; whereby upon spinal growth of a patient, the saddle or saddles as secured to the pedical or pedicles will be moved along said correction rod, so that the correction device will be automatically and synchronously adjusted for keeping pace with the spinal growth of the patient.

Nevertheless, the saddles of such a scoliosis correction device are fixed to pedicles of the spinal vertebrae by pedicle screws, which may influence the growth plates of the pedicles, thereby deferring the spinal bone growth, especially harmful for children or teenagers.

The present inventor has found the defects of the conventional arts and invented the present scoliosis correction device without hurting the growing vertebrae.

### SUMMARY OF THE INVENTION

The object of the present invention is to provide a scoliosis correction device comprising a pair of correction members suspended, hung or fastened on a patient's spinous processes, without being screwed into pedicles, whereby the vertebrae will be adjustably guided by the correction members along with the patient's growth to safely correct the patient's spine to keep at a normal restored condition.

According to the present invention, there is provided with a scoliosis correction device comprising: a first bracket having its inner side relying on a first side of a spinous process, and having a first socket formed on an outer side of the first bracket; a second bracket having its inner side relying on a second side of the spinous process and having a second socket formed on an outer side of the second socket, and having at least a fastening member provided for clamping or fastening the first socket and the second socket on both sides of the spinous process; a first guiding member connected with the first socket having at least a first guiding hole formed through the first guiding member for slidably inserting a first correction member through the first guiding hole; and a second guiding member connected with the second socket, having at least a second guiding hole formed through the second guiding member for slidably inserting a second correction member through the second guiding hole.

### BRIEF DESCRIPTION OF THE DRAWING

FIG. 1 is an exploded view of the present invention.

FIG. 2 is a perspective view of the present invention when assembled.

FIG. 3 is an elevational view of the present invention.

FIG. 4 shows a second preferred embodiment of the present invention.

FIG. 5 shows a third preferred embodiment of the present invention.

FIG. 6 is a perspective view of FIG. 5.

FIG. 7 shows a fourth preferred embodiment of the present invention.

FIG. 8 shows a fifth preferred embodiment of the present invention.

FIG. 9 shows a perspective view of the present invention.

FIG. 10 is an illustration showing the present invention as a whole system when used.

### DETAILED DESCRIPTION

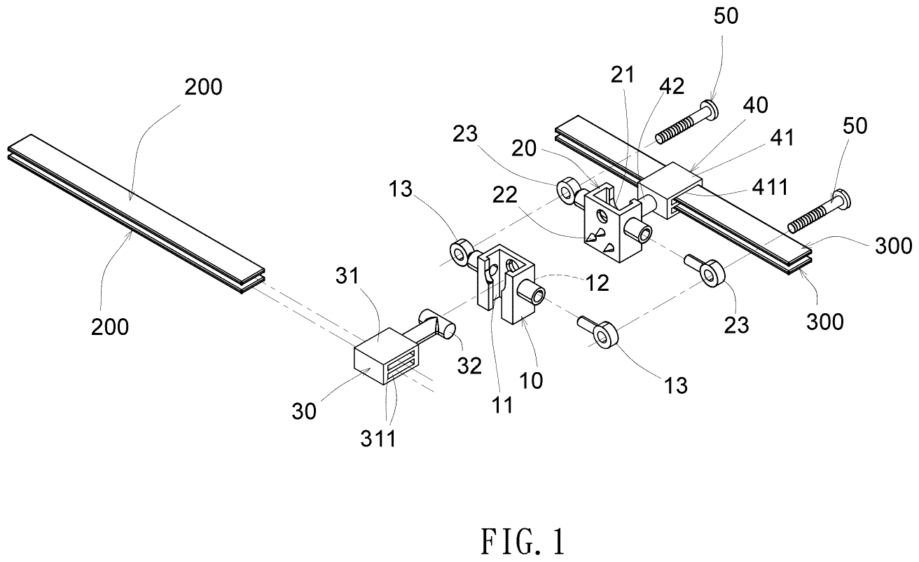

As shown in FIGS. 1˜10, the scoliosis correction device as suspended on spinous processes of the present invention comprises: a first bracket 10 having its inner side replying on a first side 101 of a spinous process 100, and having a first socket 11 formed on an outer side of the first bracket 10; a second bracket 20 having its inner side relying on a second side 102 of the spinous process 100 and having a second socket 21 formed on an outer side of the second socket 20, and having at least a fastening member 50 such as a bolt and nut provided for clamping or fastening the first bracket 10 and the second bracket 20 on both sides 101, 102 of the spinous process 100; a first guiding member 30 connected with the first socket 11, having at least a first guiding hole 311 formed through the first guiding member 30 for slidably inserting first correction member 200 through the first guiding hole 311; and a second guiding member 40 connected with the second socket 21, having at least a second guiding hole 411 formed through the second guiding member 40 for slidably inserting a second correction member 300 through the second guiding hole 411.

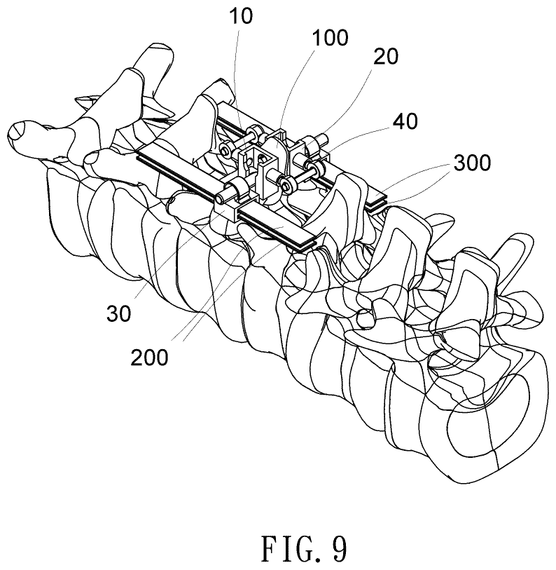

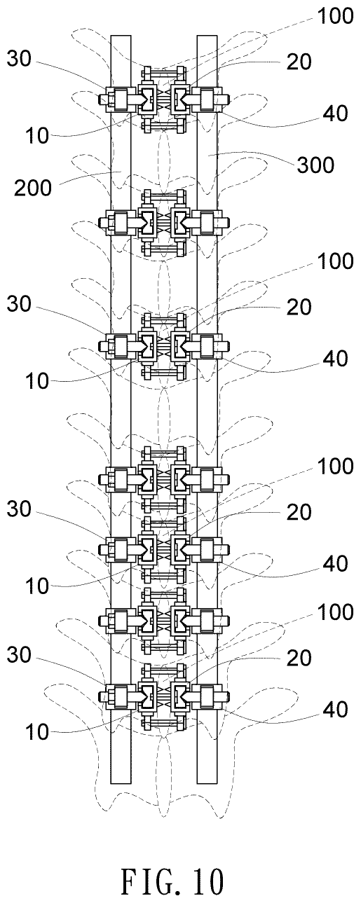

When installing the present invention as shown in FIGS. 9 and 10, the first and second brackets 10, 20 are fastened to both first and second sides of a spinous process 100, and the first and second guiding members 30, 40 are respectively connected to the first and second brackets 10, 20 for slidably inserting the first and second correction members 200, 30 through the first and second guiding members 30, 40 after the patient's spinal vertebrae are corrected to a normal or restored situation, thereby keeping the corrected spinal vertebrae at their optimum restored position as held by the two correction members 200, 300. Since the two correction members 200, 300 are suspended or hung on the spinous process 100, not fixed into the pedicles of spinal vertebrate by conventional pedicle screws, so that the growth plates of the pedicle will not be influenced. In other words, the growth of children or young patient's spine will not be influenced or retarded during their scoliosis correction treatment.

The “suspended” as used in this invention may be defined as suspended, hung, clamped or fastened to spinous processes of a patient. Namely, the scoliosis correction device of this invention may be suspended, hung, clamped or fastened on the spinous processes, rather than fixed on the pedicles of vertebrae in order not to worsely damage, hurt or influence the patient's restored spinal situation for adults or growing children, teenagers or young patients.

When applying the present invention to children, teenagers or growing young people, since the correction device of the present invention may be simultaneously moved or adjusted as slidably guided by the two correction members 200, 300 in commensuration with the patient's growth, so that repeated or frequent surgical operations for restoring, adjusting or correcting the spinal vertebrae can be prevented. It may reduce the patient's surgical complication or danger as low as possible for a better living quality and also for preventing the fear, especially for a children patient. So, the present invention provides a nice non-fusion spinal scoliosis correction device.

The correction members 200, 300 may be made of flexible or elastic materials to allow a better resilient restoring effect for a patient. The patient may have a more freedom for bending or swinging his or her body, enhancing a better living quality during the scoliosis correction period.

As shown in FIGS. 1, 3 and 4, the first bracket 10 is formed with teeth 12 on its inner side and the teeth 12 are pricked into spinous process 100 for grasping the first bracket 10 onto the process 100. The second bracket 20 is formed with teeth 22 on its inner side and the teeth 22 are pricked into process 100 for grasping the second bracket 20 onto the process 100.

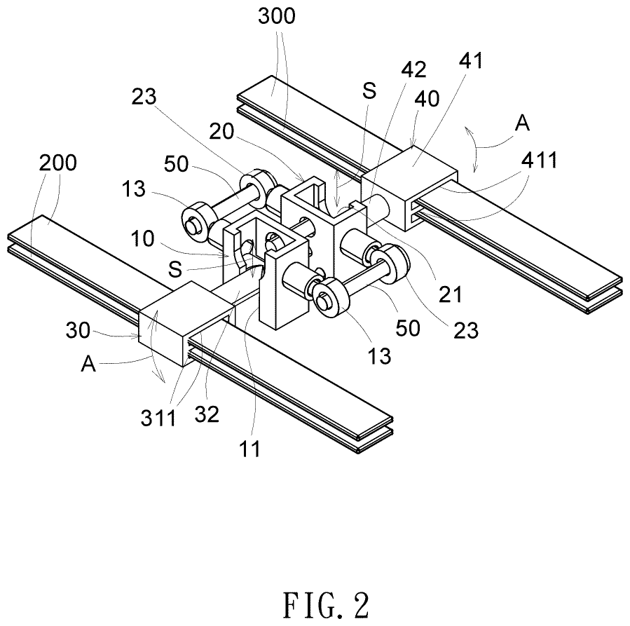

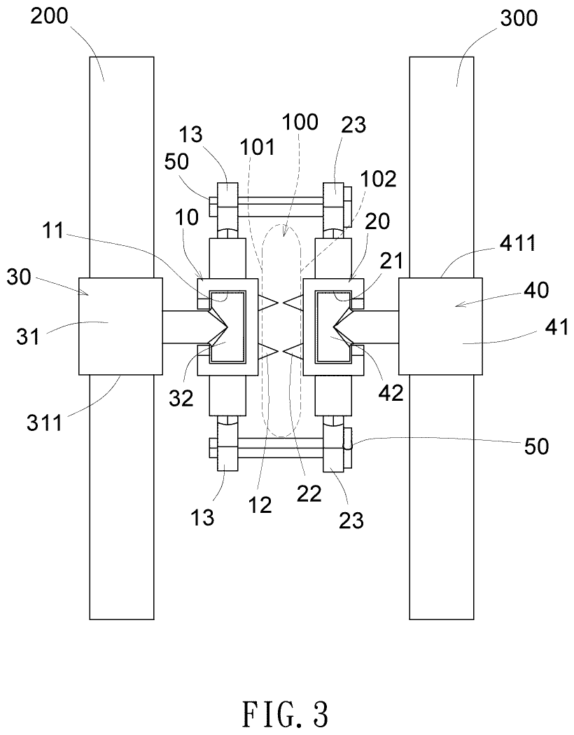

As shown in FIGS. 2 and 3, the first bracket 10 has a pair of first links 13, 13 respectively formed on an upper portion and a lower portion of the first bracket 10. The second bracket 20 has a pair of second links 23, 23 respectively formed on an upper portion and a lower portion of the second bracket 20. An upper first link 13 of the first bracket 10 is coupled to an upper second link 23 of the bracket 20 by a fastening member 50, while a lower first link 13 is coupled to a lower second link 23 by another fastening member 50, whereby upon fastening of the upper and lower fastening members 50, 50, the first bracket 10 and the second bracket 20 will be inwardly moved to be hung on the spinous process 100. The fastening member 50 may include bolt and nut, or other fastening elements.

As shown in FIG. 1˜4, the first guiding member 30 includes a first sleeve 31, having a first guiding hole 311 formed through the first sleeve 31 for slidably inserting the first correction member 200 on a left side of the process 100, and having a T-shaped first coupler 32 protruding inwardly to be frictionally held in the first socket 11 of the first bracket 10. The symbol A indicates that the guiding member 30 or 40 may be pivotally moved, while symbol S indicates the guiding member 30 or 40 may be linearly moved when installing the present invention on the spinous processes 100.

Similarly, the second guiding member 40 includes a second sleeve 41, having a second guiding hole 411 formed through the second sleeve 41 for slidably installing the second correction member 300 through the second guiding hole 411 in the second sleeve 41 at a right side of the process 100, and having a T-shaped second coupler 42 protruding inwardly from the second sleeve 41 to be frictionally held in the second socket 21 of the second bracket 20.

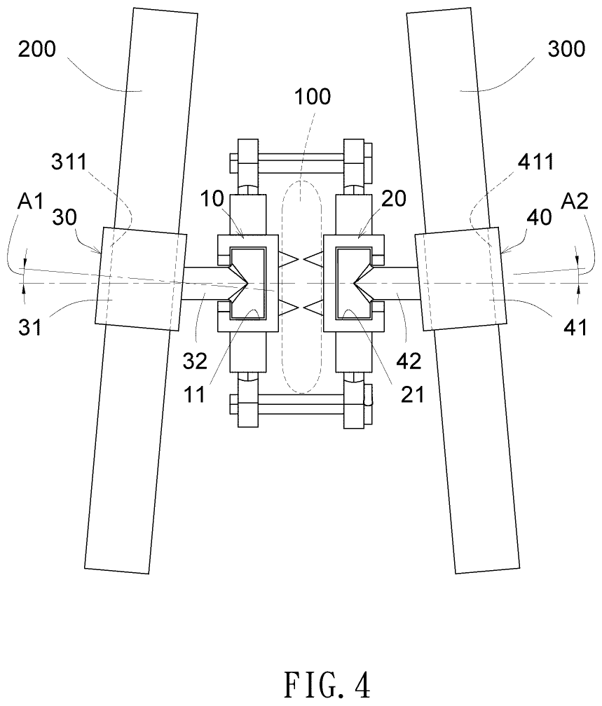

As shown in FIG. 4, the first sleeve 31 is inclinedly linked to the first coupler 32 to define an acute angle A1 between a normal line perpendicular to the first guiding hole 311 and an axis of the first coupler 32, to thereby orient the first correction member 200 inclinedly towards the spine if required by a patient. Such an acute angle is ranging from 0 to 20 degrees, optionally chosen by the doctor. Similarly, the second sleeve 41 may be inclinedly linked to the second coupler 42 for an acute angle A2 defined between a normal line perpendicular to the second guiding hole 411 and an axis of second coupler 42 so as to inclinedly orient the second correction member 300 towards the spine.

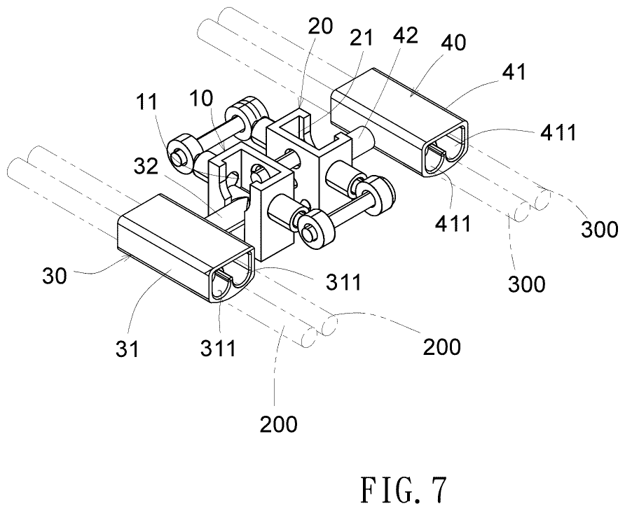

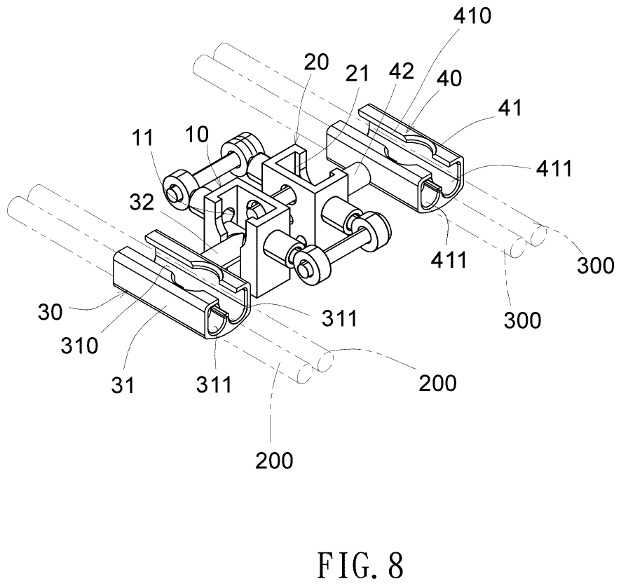

Each first guiding hole 311 or second guiding hole 411 may have a cross section of rectangular shape as shown in FIGS. 1˜6 for slidably inserting the correction members 200 or 300 within the guiding holes 311 or 411 having a cross section of rectangular shape. Other shapes may be selected. For instance, the guiding hole may be formed to have a circular cross section as shown in FIGS. 7, 8 to insert the cylindrical-shaped correction member 200 or 300. The cross sectional shapes of the correction member 200, 300 may be rectangular, circular or any other polygonal shapes, not limited in this invention.

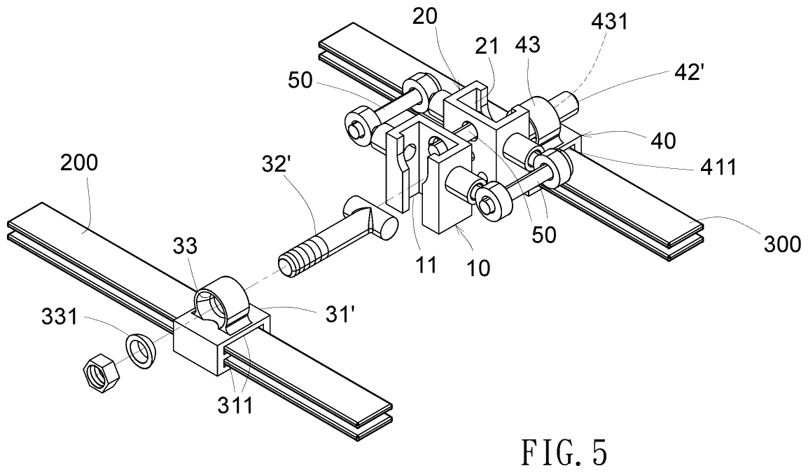

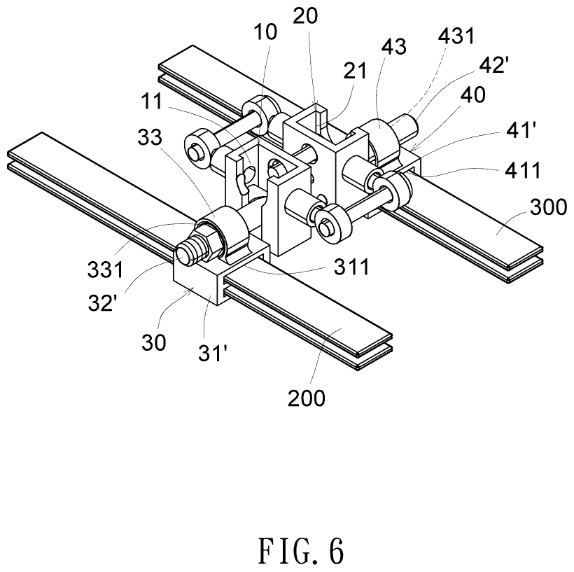

A spherical washer 331 is provided for fastening the first ball socket 33 formed on the first sleeve 31 with the first coupler 32′ protruding outwardly through the ball socket 33 by a nut as shown in FIGS. 5, 6. Similarly, another spherical washer 431 is provided for fastening the second ball socket 43 formed on the second sleeve 41 with the second coupler 42′ protruding outwardly through the ball socket 43.

As shown in FIGS. 5, 6, each sleeve 31′ or 41′ is respectively formed with a pair of guiding holes 311 or 411 as superimposedly juxtaposed for inserting a pair of correction members 200 or 300 through the pair of the guiding holes 311, 411. As shown in FIGS. 7, 8, each sleeve 31 or 41 is respectively formed with a pair of guiding holes 311, or 411 as juxtaposed side by side for inserting a pair of correction members 200 or 300 through the guiding holes 311, 411.

As shown in FIG. 8, each sleeve 31 or 41 is formed with an opening 310 or 410 to be communicated with each guiding hole 311 or 411 formed in each sleeve 31 or 41 so that each correction member 200 or 300 may be directly pressed into the guiding hole 311 or 411 through the opening 310 or 410 for a quick insertion of correction members 200, 300.

The present invention is superior to the prior art with the following advantages:
1. The correction device is suspended, hung or fastened on the spinous processes, without being fixed into pedicles of the vertebrae, for minimizing damage or hurt to the spine.
2. The vertebrae can be corrected along with the growth of a patient from time to time since the vertebrae can be slidably guidedalong the correction members of the present invention.
3. No screw is fixed into pedicle so that a growth plate of the pedicle of vertebra is not damaged or worsely influenced.

1. A scoliosis correction device comprising: a first bracket having an inner side of said first bracket relying on a first side of a spinous process, and having a first socket formed on an outer side of the first bracket; a second bracket having an inner side of said second bracket relying on a second side of the spinous process and having a second socket formed on an outer side of the second socket, and having at least a fastening member provided for clamping or fastening the first bracket and the second bracket on a right and a left side of a spinous process; a first guiding member connected with the first socket having at least a first guiding hole formed through the first guiding member for slidably inserting a first correction member through the first guiding hole; and a second guiding member connected with the second socket, having at least a second guiding hole formed through the second guiding member for slidably inserting a second correction member through the second guiding hole.

2. A device of claim 1, wherein each said bracket is formed with a plurality of teeth adapted to be pricked into a spinous process.

3. A device of claim 1, wherein said first bracket has a pair of first links respectively formed on an upper portion and a lower portion of the first bracket; and said second bracket has a pair of second links respectively formed on an upper portion and a lower portion of the second bracket, said upper first link of the first bracket coupled to an upper second link of the second bracket by a fastening member, and said lower first link coupled to said lower second link by another fastening member, whereby upon fastening of the upper and lower fastening members, the first bracket and the second bracket will be inwardly moved to be hung on the spinous process.

4. A device of claim 1, wherein the first guiding member includes a first sleeve having said first guiding hole formed through said first sleeve for slidably inserting the first correction member on a left side of the process, and having a first coupler protruding inwardly to be frictionally held in the first socket of the first bracket.

5. A device of claim 1, wherein the second guiding member includes a second sleeve having said second guiding hole formed through said second sleeve for slidably installing the second correction member at a right side of the process and having a second coupler protruding inwardly to be frictionally held in the second socket of the second bracket.

6. A device of claim 1, wherein each said sleeve is formed with said guiding hole for inserting said correction member through said guiding hole, said guiding hole having a cross sectional shape selected from rectangular, circular and polygonal shapes.

7. A device of claim 1, wherein said first guiding member includes a first sleeve having a first ball socket formed on said first sleeve, and a first spherical washer engageable with said first ball socket, and a first coupler connected to said first bracket, having an outer end of said first coupler fastened with said first ball socket as retained by said first spherical washer and a nut.

8. A device of claim 1, wherein said second guiding member includes a second sleeve having a second ball socket formed on said second sleeve, and a second spherical washer engageable with said second ball socket, and a second coupler connected to said second bracket, having an outer end of said second coupler fastened with said second ball socket as retained by said second spherical washer, and a nut.

9. A device of claim 1, wherein each said guiding member is formed with a plurality of guiding holes, adapted for inserting a plurality of said correction members through said guiding holes.

10. A device of claim 4, wherein said first sleeve is inclinedly linked to said first coupler to define an acute angle between a normal line perpendicular to the first guiding hole and an axis of the first coupler.

11. A device of claim 5, wherein said second sleeve is inclinedly linked to said second coupler to define an acute angle between a normal line perpendicular to the second guiding hole and an axis of the second coupler.
